SEQUENCE LISTING (17 sequences)

SEQ ID NO: 1 - protein, 9 aa, Artificial Sequence
  MAP3865c from aa 125 to aa 133
MIAVALAGL

SEQ ID NO: 2 - protein, 9 aa, Artificial Sequence
  MAP3865c from aa 133 to aa141
LAANFVVAL

SEQ ID NO: 3 - protein, 9 aa, Artificial Sequence
  ZnT8 from aa 178 to aa 186
MIIVSSCAV

SEQ ID NO: 4 - protein, 9 aa, Artificial Sequence
  ZnT8 from aa 186 to aa 194
VAANIVLTV

SEQ ID NO: 5 - DNA, 33 bp, Artificial Sequence
  MAP3865c gene sense primer
gcgcgaattcatgggcgccggccacaaccacac

SEQ ID NO: 6 - DNA, 34 bp, Artificial Sequence
  MAP3865c gene antisense primer
gcgcctgcagtcatcagaagctgtcggagcactc

SEQ ID NO: 7 - protein, 298 aa, Mycobacterium avium
MGAGHNHTPAETGDARLIPRMVMAAAILAAFFVVELVTSLLINSIALLADAGHMLTDVVAVFMGLAAVTLARRGSSSPARTYGWHRAEVFTAVANAGLLIGVSVFILYEAIQRLREAPAVPGVPMIAVALAGLAANFVVALLLRSHSSGSLAVKGAYLEVIADTVGSLGVLIAGVVTVTTRWPYADVVVAVLVALWVLPRAISLARDALRILSESSPTHIDVEELRAALGAVDGVTGVHDLHVWTLSPGKDMCTAHLISTGDSARVLRDARAVLSARGLAHATVQIDCPDDTECSDSF

SEQ ID NO: 8 - protein, 369 aa, Homo sapiens
MEFLERTYLVNDKAAKMYAFTLESVELQQKPVNKDQCPRERPEELESGGMYHCHSGSKPTEKGANEYAYAKWKLCSASAICFIFMIAEVVGGHIAGSLAVVTDAAHLLIDLTSFLLSLFSLWLSSKPPSKRLTFGWHRAEILGALLSILCIWVVTGVLVYLACERLLYPDYQIQATVMIIVSSCAVAANIVLTVVLHQRCLGHNHKEVQANASVRAAFVHALGDLFQSISVLISALIIYFKPEYKIADPICTFIFSILVLASTITILKDFSILLMEGVPKSLNYSGVKELILAVDGVLSVHSLHIWSLTMNQVILSAHVATAASRDSQVVRREIAKALSKSFTMHSLTIQMESPVDQDPDCLFCEDPCD

SEQ ID NO: 9 - protein, 7 aa, Artificial Sequence
  MAP3865c from aa 246 to aa 252
LSPGKDM

SEQ ID NO: 10 - protein, 7 aa, Artificial Sequence
  MAP3865c from aa 256 to aa 262
HLISTGD

SEQ ID NO: 11 - protein, 7 aa, Artificial Sequence
  MAP3865c from aa 261 ro aa 267
GDSARVL

SEQ ID NO: 12 - protein, 7 aa, Artificial Sequence
  MAP3865c from aa 281 to aa 287
HATVQID

SEQ ID NO: 13 - protein, 7 aa, Artificial Sequence
  MAP3865c from aa 121 to aa 127
PGVPMIA

SEQ ID NO: 14 - protein, 7 aa, Artificial Sequence
  MAP3865c from aa 131 to aa 137
AGLAANF

SEQ ID NO: 15 - protein, 138 aa, Artificial Sequence
  MAP3865c from aa 167 to aa 296
SLGVLIAGVVTVTTRWPYADVVVAVLVALWVLPRAISLARDALRILSESSPTHIDVEELMAPCRAALGAVDGVTGVHDLHVWTLSPGKDMCTAHLISTGDSARVLRDARAVLSARGLAHAMAPCTVQIDCPDDTECSD

SEQ ID NO: 16 - protein, 145 aa, Artificial Sequence
  ZnT8 from aa 228 to aa 366
SISVLISALIIYFKPEYKIADPICTFIFSILVLASTITILKDFSILLMEGVPKSLNYSGVZNTKELILAVDGVLSVHSLHIWSLTMNQVILSAHVATAASRDSQVVRREIAKALSKSFTMHSLZNTTIQMESPVDQDPDCLFCED

SEQ ID NO: 17 - protein, 7 aa, Artificial Sequence
  MAP3865c from aa 211 to aa 217
ILSESSP

(19) United States
(12) Patent Application Publication
(10) Pub. No.: US 2014/0271701 A1
(43) Pub. Date: Sep. 18, 2014

(54) USE OF MYCOBACTERIUM AVIUM PARATUBERCULOSIS PEPTIDES TO DIAGNOSE TYPE 1 DIABETES

(71) Applicant: UNIVERSITA' DEGLI STUDI DI SASSARI, Sassari (IT)

(72) Inventors: Leonardo Antonio Sechi, Sassari (IT); Roberto Mallone, Paris (FR)

(21) Appl. No.: 14/354,131

(22) PCT Filed: Oct. 25, 2012
(86) PCT No.: PCT/IT2012/000331

§ 371 Date: Apr. 24, 2014

(30) Foreign Application Priority Data
Oct. 26, 2011 (IT) PT105971

(51) Int. Cl. C07K 14/35; A61K 31/498; A61K 31/7048; A61K 31/439; G01N 33/569; C07K 16/12

(52) U.S. Cl. CPC C07K 14/35; G01N 33/5695; C07K 16/1289; A61K 31/7048; A61K 31/439; A61K 31/498
USPC 424/190.1; 436/501; 435/7.92; 506/9; 435/7.1; 530/326; 530/328; 530/329; 536/23.7; 435/320.1; 435/252.33; 435/287.2; 530/387.9; 514/29; 514/279; 514/250

(57) ABSTRACT

The present invention concerns antibodies recognizing Mycobacterium avium paratuberculosis epitopes able to cross-reacting with the beta-cell antigen ZnT8 to be used as early biomarkers of type 1 diabetes, epitopes for in vitro prognostic and diagnostic methods suitable to reveal a risk to develop type 1 diabetes, therapies for the prevention of T1D by avoiding, controlling or monitoring Mycobacterium paratuberculosis infection.

The present invention concerns epitopes of Mycobacterium avium paratuberculosis and antibodies recognizing thereof and cross-reacting with the beta-cell antigen znt8 as early biomarkers of type 1 diabetes.

Particularly, the present invention provide with antibodies recognizing Mycobacterium avium paratuberculosis epitopes able to cross-reacting with the beta-cell antigen ZnT8 to be used as early biomarkers of type 1 diabetes, epitopes for in vitro prognostic and diagnostic methods suitable to reveal a risk to develop type 1 diabetes, a therapy for the prevention of T1D by avoiding, controlling or monitoring Mycobacterium paratuberculosis infection.

Mycobacterium avium subspecies paratuberculosis (MAP) is transmitted from dairy herds to humans through food contamination. MAP causes an asymptomatic infection, which is highly prevalent in Sardinian T1 D patients compared with type 2 diabetes (T2D) and healthy controls. Moreover, MAP elicits humoral responses against several mycobacterial proteins.

Type 1 diabetes (T1 D) is a T cell-mediated autoimmune disease resulting from the destruction of insulin-secreting pancreatic 13 cells. It is a paradigmatic example of autoimmune disease stemming from a complex interaction between genetic and environmental factors. While several genetic susceptibility loci have been pinpointed, the environmental factors at play remain boldly elusive. Yet, environmental factors play a prominent role in T1D pathogenesis, as suggested by the incomplete (−65%) T1D concordance between monozygotic twins, by migrant studies or by the decreasing weight of susceptible and protective HLA Class II haplotypes over the last decades.

Among the environmental factors that have been called forth, viral infections—particularly enteroviruses—have received overarching attention. While epidemiological studies show a temporal correlation between enteroviral infections and appearance of anti-islet auto-antibodies (aAbs), investigations using the NOD mouse model suggest that enteroviral infections may accelerate rather than initiate T1D progression, as they are effective only once autoimmune T cells have already accumulated in the islets. The pathophysiological mechanisms through which enteroviral infections may favor T1 D development include promoting local islet inflammation, cytolytic effects on β cells and molecular mimicry. This latter concept has been proposed based on aminoacid sequence homologies and/or immune cross-reactivity between viral and β-cell epitopes.

The role of bacterial infections as T1D triggers or accelerators have received comparatively less attention. Mycobacterium avium subspecies paratuberculosis (MAP) is the causative agent of paratuberculosis (Johne's disease), a chronic enteritis that affects dairy herds. Environmental contamination with MAP is widespread, as MAP is detected in cattle's feces, soil, water (where it survives chlorination), it is shed into milk and is found in commercially pasteurized dairy preparations and meat products. Although transmitted to man, MAP infection is asymptomatic in human carriers and is not therefore regarded as a zoonosis, nor subjected to eradication in contaminated animals.

Counting ˜1.8 million inhabitants, ˜3.5 millions sheeps and approximately two hundred thousand cattle, MAP exposure may be particularly high in the Western Mediterranean island of Sardinia, where it is estimated that ˜60% of flocks may be contaminated. Sardinia is also one of the regions with the highest incidence of T1D and multiple sclerosis (MS) worldwide, a notable exception in the north-south gradient followed by these autoimmune diseases.

During the period preceding T1 D clinical onset, autoantibodies (aAbs) directed to islets antigens such as insulin, glutamic acid decarboxylase (GAD65), insulinoma associated protein-2 (IA-2) and zinc transporter 8 (Znt8) may be detectable for months up to years before disease onset and progressively wane after diagnosis. For instance, WO2008/083331 (Hutton et al.) discloses ZnT8 epitopes consisting of the C-terminal amino acids as epitopes related with the risk of T1 D development. It is also commercially available RSR ZnT8 Ab ELISA kit (RSR Limited, Avenue Park Pentwyn Cardiff CF23 8HE United Kingdom, Tel.: +44 29 2073 2076 Fax: +44 29 2073 2704 Email: info@rsrltd.com Website: www.rsrltd.com). This kit searches and identifies Abs against residues 275-369 inclusive of the human ZnT8 protein and is also capable of detecting and quantifying autoantibodies (aAbs) specific to R(Arg) 325 or to W(Trp) 325 variant or to residue Q (Glu) 325 non specific variant.

The inventors have found that Mycobacterium avium subspecies paratuberculosis (MAP) infection is a risk factor for T1D in the Sardinian population. The inventors have reported that anti-MAP and anti-ZnT8 Abs targeting homologous membrane-spanning sequences are cross-reactive and capable of eliciting strong immune responses in T1D patients, opening the possibility of a molecular mimicry mechanism precipitating disease. Particularly, the inventors have found that antibodies (Abs) against MAP3865c, which displays a sequence homology with the β-cell protein zinc transporter 8 (ZnT8), are cross-reactive with ZnT8 epitopes. Ab responses against MAP3865c were analyzed in Sardinian T1D, T2D and healthy subjects using an enzymatic immunoassay. Abs against MAP3865c recognized two immunodominant transmembrane epitopes in 52-65% of T1 D patients, but only in 5-7% of T2D and 3-5% of healthy controls. There was a linear correlation between titers of anti-MAP3865c and anti-ZnT8 Abs targeting these two homologous epitopes, and pre-incubation of sera with ZnT8 epitope peptides blocked binding to the corresponding MAP3865c peptides. These results demonstrate that Abs recognizing MAP3865c epitopes cross-react with ZnT8, underlying a molecular mimicry mechanism which may precipitate T1D in MAP-infected individuals.

MAP3865c is a 298 aminoacid 6-membrane-spanning channel which endows MAP with the ability to transport cations through the membrane, an important feature associated with intracellular survival of mycobacteria. ZnT8 is a 369 aminoacid protein which belongs to the cation diffusion facilitator family of ZnT (Slc30) proteins. It displays a remarkably similar structure and function, allowing Zn²⁺ to accumulate in the insulin granules of pancreatic β cells. Zn²⁺ cations are essential to form hexavalent insulin storage crystals and, eventually, for effective insulin secretion. Most of the 71 aminoacid difference in length between MAP3865c and ZnT8 is made up by the first extra-luminal domain, which is much shorter for MAP3865c (FIG. 3B).

To look for potential cross-reactive Ab epitopes, the analysis has been focused on a trasmembrane region of high homology. Ab reactivities against peptide sequences of this region were even more prevalent in T1D patients than those against the whole MAP3865c protein, perhaps reflecting masking of these hydrophobic epitopes in the solubilized MAP3865c protein. Importantly, Abs against this membrane-spanning epitopes would not be detected by conventional anti-ZnT8 aAb assays, which employ a fusion protein combining the 4 extra-luminal domains of ZnT8. Other regions of high homology are mostly located in these extra-luminal domains, raising the possibility that other cross-reactive epitopes may be recognized by other Abs, including conventional anti-ZnT8 aAbs. Of further note, the transmembrane region identified here does not comprise the polymorphic ZnT8 R/W variant at position 325, which is located in the last extra-luminal domain, thus making it unlikely that the ZnT8 genetic background may shape these Ab reactivities, as described for conventional anti-ZnT8 aAbs.

The intestinal localization of MAP infection may also favor cross-reactivity with Abs and T cells recognizing ZnT8. Indeed, the first encounter with β-cell antigens takes place in pancreatic lymph nodes, which also drain intestinal tissues. Epitope mimicry and spreading may be further favored by high precursor frequencies of ZnT8-reactive naïve T cells. As ZnT8 has not been found expressed by medullary thymic epithelial cells, negative selection of ZnT8-reactive T cells may be ineffective.

Thus, tolerance to ZnT8 may heavily rely on peripheral mechanisms such as immune ignorance, which may be readily overcome by MAP infection. The intestinal localization of MAP infection may also give reason for the lack of correlation between MAP IS900 DNA and Ab detection. Not all MAP-infected individuals may mount systemic Ab responses detectable in blood, or they may develop Abs against other MAP antigens.

In addition to the above, the inventors have investigated the seroreactivity against the identified ZnT8/MAP epitopes in children with new-onset T1 D and hyperglycemia (Hy) compared to healthy controls (HCs). This study shows that ZnT8/MAP peptides are recognized in new-onset T1 D. Therefore, Abs against the epitopes of the invention can be used as early markers of T1 D in pediatric population.

On the basis of the above, the present invention provides an in vitro method for diagnosing if an individual is susceptible to or is developing T1 D. The diagnostic method of the invention is more sensitive in comparison to known diagnostic methods, such as the method based on the epitopes disclosed in WO2008083331 to be performed by the commercially available RSR ZnT8 Ab ELISA kit.

The method of the invention is useful for monitoring the progression of T1D and allows to intervene in time with a treatment for preventing or delaying the onset of T1 D, for instance by avoiding, controlling or monitoring Mycobacterium paratuberculosis infection.

It is therefore specific object of the present invention the use of at least one isolated peptide belonging to MAP3865c, said at least one peptide having an homology of at least 50% in comparison to a corresponding peptide belonging to human ZnT8 sequence after optimal alignment, or at least one isolated antibody specific for said at least one peptide, as biomarker in an in vitro test for diagnosing an individual who is susceptible to or who is developing type I diabetes. Said at least one peptide can belong to MAP3865c from aminoacid 121 to aminoacid 141, preferably from aminoacid 125 to aminoacid 141, or from aminoacid 246 to aminoacid 287. Particularly, said at least one peptide can be chosen from the group consisting of MAP3865c₁₂₅₋₁₃₃ having sequence MIAVALAGL (SEQ ID NO:1), MAP3865c₁₃₃₋₁₄₁ having sequence LAANFVVAL (SEQ ID NO:2), MAP3865c₁₂₁₋₁₂₇ having sequence PGVPMIA (SEQ ID NO:13), MAP3865c₁₃₁₋₁₃₇ having sequence AGLAANF (SEQ ID NO:14), MAP3865c₂₄₆₋₂₅₂ having sequence LSPGKDM (SEQ ID NO:9), MAP3865c₂₅₆₋₂₆₂ having sequence HLISTGD (SEQ ID NO:10), MAP3865c₂₆₁₋₂₆₇ having sequence GDSARVL (SEQ ID NO:11), MAP3865c₂₈₁₋₂₈₇ having sequence HATVQID (SEQ ID NO:12). Preferably, the peptide of the invention is SEQ ID NO:1 or SEQ ID NO:12.

The invention concerns also the use of at least one isolated peptide belonging to ZnT8 sequence from aminoacid 174 to aminoacid 194, said peptide having an homology of at least 50% in comparison to a corresponding peptide belonging to MAP3865c from aminoacid 121 to aminoacid 141, or isolated antibodies specific for said at least one peptide, as biomarkers in an in vitro test for diagnosing an individual who is susceptible to or who is developing type I diabetes. Particularly, said at least one peptide can be chosen from the group consisting of ZnT8₁₇₈₋₁₈₆ having sequence MIIVSSCAV (SEQ ID NO:3), ZnT8₁₈₆₋₁₉₄ having sequence VAANIVLTV (SEQ ID NO:4), preferably ZnT8₁₇₈₋₁₈₆ having sequence MIIVSSCAV (SEQ ID NO:3).

According to an embodiment of the invention, the peptides can be at least the following four petides: MAP3865c₁₂₅₋₁₃₃ having sequence MIAVALAGL (SEQ ID NO:1), MAP3865c₁₃₃₋₁₄₁ having sequence LAANFWAL (SEQ ID NO:2), and their respective homologous peptides ZnT8₁₇₈₋₁₈₆ having sequence MIIVSSCAV (SEQ ID NO:3), ZnT8₁₈₆₋₁₉₄ having sequence VAANIVLTV (SEQ ID NO:4). According to a further embodiment of the invention the peptides are at least the following three petides: MAP3865c₁₂₅₋₁₃₃ having sequence MIAVALAGL (SEQ ID NO:1), MAP3865c₂₈₁₋₂₈₇ having sequence HATVQID (SEQ ID NO:12), ZnT8₁₇₈₋₁₈₆ having sequence MIIVSSCAV (SEQ ID NO:3).

According to a further embodiment of the invention, the peptides are all the following ten peptides: MAP3865c₁₂₅₋₁₃₃ having sequence MIAVALAGL (SEQ ID NO:1), MAP3865c₁₃₃₋₁₄₁ having sequence LAANFWAL (SEQ ID NO:2), ZnT8₁₇₈₋₁₈₆ having sequence MIIVSSCAV (SEQ ID NO:3), ZnT8₁₈₆₋₁₉₄ having sequence VAANIVLTV (SEQ ID NO:4), MAP3865c₁₂₁₋₁₂₇ having sequence PGVPMIA (SEQ ID NO:13), MAP3865c₁₃₁₋₁₃₇ having sequence AGLAANF (SEQ ID NO:14), MAP3865c₂₄₆₋₂₅₂ having sequence LSPGKDM (SEQ ID NO:9), MAP3865c₂₅₆₋₂₆₂ having sequence HLISTGD (SEQ ID NO:10), MAP3865c₂₆₁₋₂₆₇ having sequence GDSARVL (SEQ ID NO:11), MAP3865c₂₈₁₋₂₈₇ having sequence HATVQID (SEQ ID NO:12).

It is further object of the present invention a method for in vitro diagnosing a subject who is susceptible to or who is developing type I diabetes, said method comprising: a) detection and quantification, in a blood sample of said subject, of antibodies specific for at least one peptide belonging to MAP3865c, said at least one MAP3865c peptide having an homology of at least 50% in comparison to a corresponding peptide belonging to human ZnT8 sequence after optimal alignment; and/or of antibodies specific for at least one peptide belonging to ZnT8 sequence from aminoacid 174 to aminoacid 194, said ZnT8 peptide having an homology of at least 80% in comparison to a corresponding peptide belonging to MAP3865c from aminoacid 121 to aminoacid 141; b) comparison of values of step a) with those of an healthy control.

As mentioned above, said at least one peptide can belong to MAP3865c from aminoacid 121 to aminoacid 141, preferably from aminoacid 125 to aminoacid 141, or from aminoacid 246 to aminoacid 287. Particularly, said at least one peptide can be chosen from the group consisting of MAP3865c₁₂₅₋₁₃₃ having sequence MIAVALAGL (SEQ ID NO: 1), MAP3865c₁₃₃₋₁₄₁ having sequence LAANFVVAL (SEQ ID NO:2), MAP3865c₁₂₁₋₁₂₇ having sequence PGVPMIA (SEQ ID NO:13), MAP3865c₁₃₁₋₁₃₇ having sequence AGLAANF (SEQ ID NO:14), MAP3865c₂₄₆₋₂₅₂ having sequence LSPGKDM (SEQ ID NO:9), MAP3865c₂₅₆₋₂₆₂ having sequence HLISTGD (SEQ ID NO:10), MAP3865c₂₆₁₋₂₆₇ having sequence GDSARVL (SEQ ID NO:11), MAP3865c₂₈₁₋₂₈₇ having sequence HATVQID (SEQ ID NO:12), ZnT8₁₇₈₋₁₈₆ having sequence MIIVSSCAV (SEQ ID NO:3), ZnT8₁₈₆₋₁₉₄ having sequence VAANIVLTV (SEQ ID NO:4). Preferably SEQ ID NO:1, SEQ ID NO:12 or SEQ ID NO:3.

According to an embodiment of the invention, the peptides are at least the following four peptides: MAP3865c₁₂₅₋₁₃₃ having sequence MIAVALAGL (SEQ ID NO:1), MAP3865c₁₃₃₋₁₄₁ having sequence LAANFWAL (SEQ ID NO:2), and their respective homologous peptides ZnT8₁₇₈₋₁₈₆ having sequence MIIVSSCAV (SEQ ID NO:3), ZnT8₁₈₆₋₁₉₄ having sequence VAANIVLTV (SEQ ID NO:4). According to a further embodiment of the invention the above method is based on at least the following three peptides: MAP3865c₁₂₅₋₁₃₃ having sequence MIAVALAGL (SEQ ID NO:1), MAP3865c₂₈₁₋₂₈₇ having sequence HATVQID (SEQ ID NO:12), ZnT8₁₇₈₋₁₈₆ having sequence MIIVSSCAV (SEQ ID NO:3). An embodiment of the invention provide also the above method wherein the peptides are all the following ten peptides: MAP3865c₁₂₅₋₁₃₃ having sequence MIAVALAGL (SEQ ID NO:1), MAP3865c₁₃₃₋₁₄₁ having sequence LAANFWAL (SEQ ID NO:2), ZnT8₁₇₈₋₁₈₆ having sequence MIIVSSCAV (SEQ ID NO:3), ZnT8₁₈₆₋₁₉₄ having sequence VAANIVLTV (SEQ ID NO:4), MAP3865c₁₂₁₋₁₂₇ having sequence PGVPMIA (SEQ ID NO:13), MAP3865c₁₃₁₋₁₃₇ having sequence AGLAANF (SEQ ID NO:14), MAP3865c₂₄₆₋₂₅₂ having sequence LSPGKDM (SEQ ID NO:9), MAP3865c₂₅₆₋₂₆₂ having sequence HLISTGD (SEQ ID NO:10), MAP3865c₂₆₁₋₂₆₇ having sequence GDSARVL (SEQ ID NO:11), MAP3865c₂₈₁₋₂₈₇ having sequence HATVQID (SEQ ID NO:12). The method according to the present invention can be carried out by ELISA.

In addition, the present concerns a method for in vitro diagnosing a subject who is susceptible to or who is developing type I diabetes, said method comprising incubating a blood sample comprising lymphocytes from said subject in the presence of at least one peptide belonging to MAP3865c, said at least one MAP3865c peptide having an homology of at least 50% in comparison to a corresponding peptide belonging to human ZnT8 sequence after optimal alignment; and/or in the presence of at least one peptide belonging to ZnT8 sequence from aminoacid 174 to aminoacid 194, said ZnT8 peptide having an homology of at least 80% in comparison to a corresponding peptide belonging to MAP3865c from aminoacid 121 to aminoacid 141, for a time and under conditions sufficient to stimulate the lymphocytes to produce an effector molecule, such as interferon-γ, a cytokine, an interleukin and/or TNF-α, wherein the presence or level of the effector molecule is indicative of the lymphocytes derived from a subject susceptible to or who is developing type I diabetes. According to the above method, said at least one peptide belongs to MAP3865c from aminoacid 121 to aminoacid 141, preferably from aminoacid 125 to aminoacid 141, or from aminoacid 246 to aminoacid 287. Particularly, said at least one peptide is chosen from the group consisting of MAP3865c₁₂₅₋₁₃₃ having sequence MIAVALAGL (SEQ ID NO:1), MAP3865c₁₃₃₋₁₄₁ having sequence LAANFWAL (SEQ ID NO:2), MAP3865c₁₂₁₋₁₂₇ having sequence PGVPMIA (SEQ ID NO:13), MAP3865c₁₃₁₋₁₃₇ having sequence AGLAANF (SEQ ID NO:14), MAP3865c₂₄₆₋₂₅₂ having sequence LSPGKDM (SEQ ID NO:9), MAP3865c₂₅₆₋₂₆₂ having sequence HLISTGD (SEQ ID NO:10), MAP3865c₂₆₁₋₂₆₇ having sequence GDSARVL (SEQ ID NO:11), MAP3865c₂₈₁₋₂₈₇ having sequence HATVQID (SEQ ID NO:12), ZnT8₁₇₈₋₁₈₆ having sequence MIIVSSCAV (SEQ ID NO:3), ZnT8₁₈₆₋₁₉₄ having sequence VAANIVLTV (SEQ ID NO:4), preferably SEQ ID NO:1, SEQ ID NO:12 or SEQ ID NO:3. According to an embodiment of the invention, the peptides are at least the following four peptides: MAP3865c₁₂₅₋₁₃₃ having sequence MIAVALAGL (SEQ ID NO:1), MAP3865c₁₃₃₋₁₄₁ having sequence LAANFWAL (SEQ ID NO:2) and their respective homologous peptides ZnT8₁₇₈₋₁₈₆ having sequence MIIVSSCAV (SEQ ID NO:3), ZnT8₁₈₆₋₁₉₄ having sequence VAANIVLTV (SEQ ID NO:4). According to a further embodiment of the invention, the peptides are at least the following three peptides: MAP3865c₁₂₅₋₁₃₃ having sequence MIAVALAGL (SEQ ID NO:1), MAP3865c₂₈₁₋₂₈₇ having sequence HATVQID (SEQ ID NO:12), ZnT8₁₇₈₋₁₈₆ having sequence MIIVSSCAV (SEQ ID NO:3). According to a further embodiment of the invention, the method can use all the following ten peptides: MAP3865c₁₂₅₋₁₃₃ having sequence MIAVALAGL (SEQ ID NO:1), MAP3865c₁₃₃₋₁₄₁ having sequence LAANFVVAL (SEQ ID NO:2), ZnT8₁₇₈₋₁₈₆ having sequence MIIVSSCAV (SEQ ID NO:3), ZnT8₁₈₆₋₁₉₄ having sequence VAANIVLTV (SEQ ID NO:4), MAP3865c₁₂₁₋₁₂₇ having sequence PGVPMIA (SEQ ID NO:13), MAP3865c₁₃₁₋₁₃₇ having sequence AGLAANF (SEQ ID NO:14), MAP3865c₂₄₆₋₂₅₂ having sequence LSPGKDM (SEQ ID NO:9), MAP3865c₂₅₆₋₂₆₂ having sequence HLISTGD (SEQ ID NO:10), MAP3865c₂₆₁₋₂₆₇ having sequence GDSARVL (SEQ ID NO:11), MAP3865c₂₈₁₋₂₈₇ having sequence HATVQID (SEQ ID NO:12).

The present invention concerns an isolated peptide belonging to MAP3865c, said MAP3865c peptide having an homology of at least 50% in comparison to a corresponding peptide belonging to human ZnT8 sequence after optimal alignment; or belonging to ZnT8 sequence from aminoacid 174 to aminoacid 194, said ZnT8 peptide having an homology of at least 80% in comparison to a corresponding peptide belonging to MAP3865c from aminoacid 121 to aminoacid 141. Particularly, the isolated peptide can belong to MAP3865c from aminoacid 121 to aminoacid 141, preferably from aminoacid 125 to aminoacid 141, or from aminoacid 246 to aminoacid 287. Preferably, isolated peptide is chosen from the group consisting of MAP3865c₁₂₅₋₁₃₃ having sequence MIAVALAGL (SEQ ID NO:1), MAP3865c₁₃₃₋₁₄₁ having sequence LAANFVVAL (SEQ ID NO:2), MAP3865c₁₂₁₋₁₂₇ having sequence PGVPMIA (SEQ ID NO:13), MAP3865c₁₃₁₋₁₃₇ having sequence AGLAANF (SEQ ID NO:14), MAP3865c₂₄₆₋₂₅₂ having sequence LSPGKDM (SEQ ID NO:9), MAP3865c₂₅₆₋₂₆₂ having sequence HLISTGD (SEQ ID NO:10), MAP3865c₂₆₁₋₂₆₇ having sequence GDSARVL (SEQ ID NO:11), MAP3865c₂₈₁₋₂₈₇ having sequence HATVQID (SEQ ID NO:12), ZnT8₁₇₈₋₁₈₆ having sequence MIIVSSCAV (SEQ ID NO:3), ZnT8₁₈₆₋₁₉₄ having sequence VAANIVLTV (SEQ ID NO:4), preferably SEQ ID NO:1, SEQ ID NO:12 or SEQ ID NO:3.

In addition, the present invention concerns an isolated nucleic acid molecule encoding for the peptide as defined above, a vector comprising said nucleic acid molecule, an isolated cell comprising said vector.

The present invention concerns also a kit comprising a container, said container comprising at least one peptide as defined above or at least one nucleic acid molecule as defined above. The kit peptides can be at least the following four peptides: MAP3865c₁₂₅₋₁₃₃ having sequence MIAVALAGL (SEQ ID NO:1), MAP3865c₁₃₃₋₁₄₁ having sequence LAANFVVAL (SEQ ID NO:2), and their respective homologous peptides ZnT8₁₇₈₋₁₈₆ having sequence MIIVSSCAV (SEQ ID NO:3), ZnT8₁₈₆₋₁₉₄ having sequence VAANIVLTV (SEQ ID NO:4). In addition, the kit peptides can be at least the following three peptides: MAP3865c₁₂₅₋₁₃₃ having sequence MIAVALAGL (SEQ ID NO:1), MAP3865c₂₈₁₋₂₈₇ having sequence HATVQID (SEQ ID NO:12), ZnT8₁₇₈₋₁₈₆ having sequence MIIVSSCAV (SEQ ID NO:3).

Preferably, the kit of the invention can comprose all the following ten peptides: MAP3865c₁₂₅₋₁₃₃ having sequence MIAVALAGL (SEQ ID NO:1), MAP3865c₁₃₃₋₁₄₁ having sequence LAANFWAL (SEQ ID NO:2), ZnT8₁₇₈₋₁₈₆ having sequence MIIVSSCAV (SEQ ID NO:3), ZnT8186-194 having sequence VAANIVLTV (SEQ ID NO:4), MAP3865c₁₂₁₋₁₂₇ having sequence PGVPMIA (SEQ ID NO:13), MAP3865c₁₃₁₋₁₃₇ having sequence AGLAANF (SEQ ID NO:14), MAP3865c₂₄₆₋₂₅₂ having sequence LSPGKDM (SEQ ID NO:9), MAP3865c₂₅₆₋₂₆₂ having sequence HLISTGD (SEQ ID NO:10), MAP3865c₂₆₁₋₂₆₇ having sequence GDSARVL (SEQ ID NO:11), MAP3865c₂₈₁₋₂₈₇ having sequence HATVQID (SEQ ID NO:12).

The present invention concerns also an isolated antibody specific for the peptide as defined above.

It is further object of the present invention, a vaccine for the treatment or prophylaxis of type I diabetes, said vaccine comprising or consisting of at least one isolated peptide as defined above. Preferably SEQ ID NO:1, SEQ ID NO:12 or SEQ ID NO:3. The vaccine peptides can be at least the following four peptides: MAP3865c₁₂₅₋₁₃₃ having sequence MIAVALAGL (SEQ ID NO:1), MAP3865c₁₃₃₋₁₄₁ having sequence LAANFWAL (SEQ ID NO:2), and their respective homologous peptides ZnT8₁₇₈₋₁₅₆ having sequence MIIVSSCAV (SEQ ID NO:3), ZnT8₁₈₆₋₁₉₄ having sequence VAANIVLTV (SEQ ID NO:4). According to a further embodiment of the invention, vaccine peptides are at least the following three peptides: MAP3865c₁₂₅₋₁₃₃ having sequence MIAVALAGL (SEQ ID NO:1), MAP3865c₂₈₁₋₂₈₇ having sequence HATVQID (SEQ ID NO:12), ZnT8₁₇₈₋₁₈₆ having sequence MIIVSSCAV (SEQ ID NO:3).

Preferably, vaccine peptides are all the following ten peptides: MAP3865c₁₂₅₋₁₃₃ having sequence MIAVALAGL (SEQ ID NO:1), MAP3865c₁₃₃₋₁₄₁ having sequence LAANFVVAL (SEQ ID NO:2), ZnT8₁₇₈₋₁₈₆ having sequence MIIVSSCAV (SEQ ID NO:3), ZnT8₁₈₆₋₁₉₄ having sequence VAANIVLTV (SEQ ID NO:4), MAP3865c₁₂₁₋₁₂₇ having sequence PGVPMIA (SEQ ID NO:13), MAP3865c₁₃₁₋₁₃₇ having sequence AGLAANF (SEQ ID NO:14), MAP3865c₂₄₆₋₂₅₂ having sequence LSPGKDM (SEQ ID NO:9), MAP3865c₂₅₆₋₂₆₂ having sequence HLISTGD (SEQ ID NO:10), MAP3865c₂₆₁₋₂₆₇ having sequence GDSARVL (SEQ ID NO:11), MAP3865c₂₈₁₋₂₈₇ having sequence HATVQID (SEQ ID NO:12). It is further object of the present invention anti Mycobacterium avium paratuberculosis drugs, such as for example clarithromycin, rifabutin, clofazimine, for use in the prevention and treatment of type I diabetes.

The present invention now will be described by an illustrative, but not limitative way, according to preferred embodiments thereof, with particular reference to enclosed drawings wherein:

FIG. 1. Prevalence of anti-MAP3865c Abs in Sardinian T1D and T2D patients. Sera were tested for their reactivity against plate-coated MAP3865c-MBP fusion protein. Ab distribution is shown for T1D (A) and T2D (B) patients compared to healthy controls. Dotted lines indicate the cut-off for positivity used in each assay, as calculated by ROC analysis. The percent fraction of Ab+ sera is indicated on top of each distribution, while bars indicate the corresponding median±interquartile range. AUC and p values are given in the top right corner. Figures show representative experiments out of three performed.

FIG. 2. (A) Reactivity against the MAP-MBP fusion protein is MAP-specific. Ab+ and Ab-negative sera from T1D and healthy donors were challenged either with the MAP-MBP fusion protein (as in FIG. 1) or with a LacZ-MBP control protein. The dotted line indicates the cutoff for positivity. (B) Intra- and inter-assay variability of MAP3865c ELISA Ab assays. For intra-assay variability (white bars), the same serum was tested in 20 replicate wells; bars show readouts of each single well. CV is 2.8%. For inter-assay variability (grey bars), the same serum was tested in 4 separate experiments; bars show mean±SEM of triplicate wells from each experiment. CV is 7.4%.

FIG. 3. (A) Aminoacid sequence alignment of ZnT8 (Slc30A8) and MAP3865c proteins. Conserved aminoacid residues are highlighted in grey within the MAP3865c sequence and listed in bold below the two sequence alignment rows. The other highlighted parts refer to the ZnT8 protein structure shown in (B): sequences highlighted in bold type belong to the 3 intra-luminal loops; the sequence in not dotted rectangle belongs to the fourth transmembrane domain, while those underlined belong to the other transmembrane regions; sequences not highlighted fall within the 4 extra-luminal fragments. Dotted rectangles show the MAP3865c₁₂₅₋₁₃₃/ZnT8₁₇₈₋₁₈₆ and MAP3865c₁₃₃₋₁₄₁/ZnT8₁₈₆₋₁₉₄ peptides studied in subsequent experiments. The topology of the ZnT8 protein is also shown in panel (B), where the 3 intra-luminal loops become extracellularly exposed once the insulin granule is released. Conversely, the 4 extra-luminal domains are exposed to the cytosol and remain intracellular upon insulin exocytosis.

FIG. 4. Ab reactivities against the MAP3865c protein are inhibited by MAP3865c₁₂₅₋₁₃₃ and MAP3865c₁₃₃₋₁₄₁ peptides. Ab+ and Ab− negative sera from T1D patients were pre-incubated overnight with saturating concentrations (5.5 μM) of MAP3865c₁₂₅₋₁₃₃, MAP3865c₁₃₃₋₁₄₁, the two peptides in combination, MAP3865c-MBP fusion protein and control or no peptide. Their reactivity on MAP3865c-MBP-coated ELISA plates was subsequently tested. Bars depict means±SEM of triplicate wells and results are representative of three separate experiments.

FIG. 5. Prevalence of Abs against MAP3865c₁₂₅₋₁₃₃ (A) and its homologous ZnT8₁₇₈₋₁₈₆ (B); and against MAP3865c₁₃₃₋₁₄₁ (C) and its homologous ZnT8₁₈₆₋₁₉₄ (D) in T1D and healthy subjects. Data representation is the same as in FIG. 1.

FIG. 6. Prevalence of Abs against MAP3865c₁₂₅₋₁₃₃ (A) and its homologous ZnT8₁₇₈₋₁₈₆ (B); and against MAP3865c₁₃₃₋₁₄₁ (C) and its homologous ZnT8₁₈₆₋₁₉₄ (D) in T2D and healthy subjects. Data representation is the same as in FIG. 1.

FIG. 7. Correlation between titers of MAP3865c- and ZnT8-reactive Abs recognizing different epitopes. Correlations are shown between titers of Abs recognizing (A) MAP3865c₁₂₅₋₁₃₃ and its homologous ZnT8₁₇₈₋₁₈₆ epitope; (B) MAP3865c₁₃₃₋₁₄₁ and its homologous ZnT8₁₈₆₋₁₉₄ epitope; (C) MAP3865c₁₂₅₋₁₃₃ and its consecutive MAP3865c₁₃₃₋₁₄₁ epitope; (D) ZnT8₁₇₈₋₁₈₆ and its consecutive ZnT8₁₈₆₋₁₉₄ epitope. Each circle represents the titers of one T1 D or healthy donor.

FIG. 8. Ab reactivities against MAP3865c epitopes are inhibited by the homologous ZnT8 epitopes. (A) Ab+ and Ab− negative sera from T1D patients were pre-incubated overnight with saturating concentrations of MAP3865c₁₂₅₋₁₃₃ (white bars), ZnT8₁₇₈₋₁₈₆ (hatched bars), control (grey bars) or no peptide (black bars) and their reactivity on MAP3865c₁₂₅₋₁₃₃-coated ELISA plates subsequently tested. (B) The same sera were preincubated with MAP3865c₁₃₃₋₁₄₁ (white bars), ZnT8₁₈₆₋₁₉₄ (hatched bars), control (grey bars) or no peptide (black bars) and their reactivity on MAP3865c₁₃₃₋₁₄₁-coated ELISA plates subsequently tested. Bars depict means±SEM of triplicate wells and results are representative of two separate experiments.

FIG. 9. Prevalence of Abs against homologous ZnT8 and MAP3865c epitopes in 31 Type 1 Diabetes (T1D), and 30 healthy controls (HCs) Sardinian adult. Sera were tested for their reactivity against plate-coated with MAP3865c₁₂₅₋₁₃₃/ZnT8₁₇₈₋₁₈₆ (A)/(B) and MAP3865c₁₃₃₋₁₄₁/ZnT8₁₅₆₋₁₉₄ (C)/(D) homologous peptides. Figure show representative experiments out of three performed.

FIG. 10. Prevalence of Abs against MAP3865c epitopes falling into the region of homology comprising the polymorphic Znt8 325th residue in T1D and HCs Sardinian adult. Sera were tested for their reactivity against plate-coated with MAP3865c₂₄₆₋₂₅₂(A) MAP3865c₂₅₆₋₂₆₂ (B) MAP3865c₂₆₁₋₂₆₇ (C) and MAP3865c₂₈₁₋₂₈₇ (D) peptides. Figure show representative experiments out of three performed.

FIG. 11. Prevalence of Abs against the C-terminal region of human Znt8 targeted by ElisaRSR™ ZnT8 Ab™kit. The horizontal black line represents the cut off value of 15u/ml.

FIG. 12. Prevalence of Abs against homologous ZnT8 and MAP3865c epitopes in Type 1 Diabetes (T1D), and healthy controls (HCs) Sardinian children. Sera were tested for their reactivity against plate-coated with MAP3865c₁₃₃₋₁₄₁(A) ZnT8₁₅₆₋₁₉₄ (B) homologous peptides. Figure show representative experiments out of three performed

FIG. 13. Prevalence of Abs against MAP3865c epitopes falling into the region of homology comprising the polymorphic Znt8 325th residue in 29 T1D and 30 HCs Sardinian children. Sera were tested for their reactivity against plate-coated with MAP3865c₂₄₆₋₂₅₂(A) MAP3865c₂₅₆₋₂₆₂ (B) MAP3865c₂₆₁₋₂₆₇ (C) and MAP3865c₂₈₁₋₂₈₇ (D) peptides. Figure show representative experiments out of three performed.

### EXAMPLE 1

### Study on the Cross Reactivity Between Antibodies Recognizing Mycobacterium avium Paratuberculosis Epitopes and Beta-Cell Antigen Znt8 in Type 1 Diabetes Patients

Methods

Patient and Control Serum Samples

T1D patients (n=34; mean age 34.5±7.7 years, mean age at onset 17.5±10.2 years, mean T1D duration 16.8±9.9 years) and T2D patients (n=56; mean age 64.8±8.6 years, mean age at onset 56.4±9.2 years, mean T2D duration 8.5±5.3 years) diagnosed according to the American Association of Diabetes criteria and healthy blood donors (n=63) age-matched with T1D patients (mean age 38.5±12.0 years; p=0.102) were recruited at the University Hospital of Sassari. Written informed consents were obtained before blood drawing and the study was approved by the University Ethics Committee. Serum samples were processed as previously described.

Construction of the pMAL-MAP3865c Expression Vector

MAP DNA was extracted with the detergent cetyltrimethylammonium bromide (Sigma). The fulllength MAP3865c gene was amplified by PCR from the MAP DNA ATCC43015 with a sense primer (5′-GCGCGAATTCATGGGCGCCGGCCACAACCACAC-3′) (SEQ ID NO:5) and an antisense primer (5′-GCGCCTGCAGTCATCAGAAGCTGTCGGAGCACTC-3′) (SEQ ID NO:6), where sequences are EcoRI and PstI restriction sites, respectively. The MAP3865c coding sequence was cloned into pMALc2X (New England Biolabs) next to a maltose-binding protein (MBP) sequence, and the ligation mix was used to transform E. coli K12 TB1 competent cells. Transformants were screened by plating the electroporated K12 TB1 cells on rich medium (10 g/l tryptone, 5 g/l yeast extract, 5 g/l NaCl) supplemented with ampicillin (100 μg/ml). The coding sequence of the cloned MAP3865c gene fully matched the published sequence of the MAP3865c gene of M. paratuberculosis K10 (GenBank accession number: NC002944).

MAP3865c Protein Expression and Purification

E. coli TB1 cells harboring the expression plasmid were grown at 37° C. and a single colony was used to inoculate rich medium containing 1 g/l ampicillin and 2 g/l glucose. MAP3865c-MBP fusion protein expression was induced by addition of 0.3 mM isopropyl-β-D-thiogalactopyranoside (Sigma). After 2 h, cells were harvested, resuspended in 20 ml of column buffer (20 mM Tris-HCl, 200 mM NaCl, 1 mM ethylenediaminetetraacetic acid (EDTA), 1:100 Sigma protease inhibitor cocktail) and frozen at −20° C. The following day, cells were lysed by sonication. Debris were removed by centrifugation, supernatants were diluted 1:5 with column buffer, loaded on a column charged with amylose resin (New England Biolabs) and washed 5 times. The fusion protein was eluted with column buffer containing 10 mM maltose. The MAP3865c-MBP fusion protein migrated at the expected molecular mass of 72.5 kD in sodium dodecyl sulfate polyacrylamide gel electrophoresis.

Peptides

Peptides MAP3865c₁₂₅₋₁₃₃ (MIAVALAGL) (SEQ ID NO: 1) and MAP3865c₁₃₃₋₁₄₁ (LAANFWAL) (SEQ ID NO:2) along with their respective homologous peptides ZnT8₁₇₈₋₁₈₆ (MIIVSSCAV) (SEQ ID NO:3) and ZnT8₁₈₆₋₁₉₄ (VAANIVLTV) (SEQ ID NO:4) were synthesized at >85% purity

(GL Biochem). Conserved aminoacid residues are underlined. Stock solutions (10 mM in dimethyl sulfoxide) were stored in single-use aliquots at −80° C.

Enzymatic Immunoassay

An indirect enzyme-linked immunosorbent assay (ELISA) were set up to detect Abs specific for MAP3865c protein and peptides. Ninety-six-well Nunc immunoplates were coated overnight at 4° C. with 5 μg/ml of recombinant MAP3865c-MBP fusion protein or 10 μg/ml of peptides diluted in 0.05 M carbonate-bicarbonate buffer, pH 9.5 (Sigma). Plates were then blocked for 1 h at room temperature with 5% non-fat dried milk (Sigma) and washed twice with phosphate-buffered saline (PBS) containing 0.05% Tween-20 (PBS-T). Serum samples were subsequently added at 1:100 dilution in PBS-T for 2 h at room temperature. After 5 washes in PBS-T, 100 μl of alkaline phosphatase-conjugated goat anti-human immunoglobulin G polyclonal Ab (1:1000; Sigma) was added for 1 h at room temperature. Plates were washed again 5 times in PBS-T and paranitrophenylphosphate (Sigma) added as substrate for alkaline phosphatase. Plates were incubated at 37° C. in the dark for 3-6 min and the absorbance at 405 nm read on a VERSATunable Max microplate reader (Molecular Devices). Negative control wells were obtained by incubation of immobilized protein or peptides with secondary Ab alone, and their mean values subtracted from all samples. Positive control sera were also included in all experiments. Results are expressed as means of triplicate 405 nm optical density (OD) values.

Competition Assays

Competition assays were performed by pre-incubating sera overnight at 4° C. with saturating concentrations (5-20 μM, titrated for each individual serum) of MAP peptides, the corresponding ZnT8 peptides, irrelevant peptide (MAP3865c₂₁₁₋₂₁₇, ILSESSP (SEQ ID NO:17)), no peptide, or MAP3865c-MBP fusion protein, as previously described. Sera were then subjected to ELISA on plates coated with MAP3865c-MBP, MAP3865c₁₂₅₋₁₃₃ or MAP3865c₁₃₃₋₁₄₁, as above.

MAP IS900 PCR

The presence of MAP-specific DNA in blood samples was detected by PCR amplification of IS900 sequences, as previously described.

Statistical Analyses

Receiver operator characteristic (ROC) curves were used to score the performance of each single ELISA in discriminating T1D or T2D patients from healthy controls. AUC was calculated assuming a non-parametric distribution of results. Thus, an AUC of 1.0 would indicate that the assay achieved 100% accuracy in identifying patients; an AUC of 0.5 would indicate that the assay gave no difference between patients and controls; and an AUC of 0 would indicate that the assay gave a positive result for controls and a negative result for patients. The cut-off for positivity in each assay was set at ≧93% specificity (i.e. Ab+ healthy controls ≦7%) and the corresponding sensitivity (i.e. percent of Ab+ patients) calculated accordingly. Clinical characteristics of Ab+ and Ab− negative patients were compared using the Mann-Whitney U test.

Results

Anti-MAP3865c Abs are highly prevalent in Sardinian T1D patients, but not in T2D patients. The purified MAP3865c-MBP fusion protein was first used to screen by ELISA for the presence of serum anti-MAP3865c Abs. As shown in FIG. 1A, 29.4% of T1D patients displayed serum reactivity against MAP3865c compared to 6.4% of healthy controls (AUC 0.68, p=0.014). This reactivity was specific of T1 D patients, as it was not significantly different between T2D patients and controls (FIG. 1B; 3.6% vs 2.9%; AUC 0.55; p=0.396).

Since the MAP3865c protein was fused with MBP, Ab+ and Ab-negative sera were tested against the LacZ-MBP control to exclude potential MBP-specific reactivities. A difference in Ab reactivity between Ab+ and Ab− negative sera and between T1D and healthy subjects was only observed when testing with the MAP-MBP protein, while the LacZ MBP protein did not discriminate any positive sample using the same sera (FIG. 2A).

The ELISA assay employed displayed good reproducibility. For determination of intra-assay variability, a serum with MAP3865c Ab reactivity near the cut-off values was tested 20 times in a single experiment, giving a coefficient of variation (CV) of 2.8%. (FIG. 2B). The same serum tested in 4 separate experiments yielded an inter-assay CV of 7.4% (FIG. 2B).

As previously reported, the presence of MAP-specific IS900 DNA was also more prevalent among T1D patients (55.9%) than among T2D and healthy controls (7.0% and 20.0%, respectively; p<0.001). However, there was no correlation between positivity for anti-MAP3865c Abs and IS900 DNA (Table 1), although the frequency of Ab+T1D patients was higher in the IS900 DNA+ group (7/34 20.6% vs 3/34, 8.8%).

Table 1 shows the prevalence of MAP-specific IS900 DNA and of anti-MAP3865c Abs in the peripheral blood of T1 D patients (n=34).

TABLE 1

| | IS900 PCR+ | IS900 PCR− |
|---|---|---|
| MAP3865c Ab+ | 20.6% | 8.8% |
| MAP3865c Ab− | 52.9% | 17.7% |

Anti-MAP3865c Abs Recognize an Immunodominant Transmembrane Region Homologous to ZnT8.

Scanning of the MAP3865c aminoacid sequence unraveled a 27.5% sequence identity with the human β-cell protein ZnT8 (Slc30A8) (FIG. 3A).

FIG. 3 shows aminoacid sequence alignment of ZnT8 and MAP3865c proteins.

MAP3865c protein has the following sequence (SEQ ID NO:7):

NCBI Reference Sequence: NP_962799.1
>gi|41409963|ref|NP_962799.1| MAP3865c
MGAGHNHTPAETGDARLIPRMVMAAAILAAFFVVELVTSLLINSIALLAD
AGHMLTDVVAVFMGLAAVTLARRGSSSPARTYGWHRAEVFTAVANAGLLI
GVSVFILYEAIQRLREAPAVPGVPMIAVALAGLAANFVVALLLRSHSSGS
LAVKGAYLEVIADTVGSLGVLIAGVVTVTTRWPYADVVVAVLVALWVLPR
AISLARDALRILSESSPTHIDVEELRAALGAVDGVTGVHDLHVWTLSPGK
DMCTAHLISTGDSARVLRDARAVLSARGLAHATVQIDCPDDTECSDSF

ZnT8 protein has the following sequence (SEQ ID NO:8):

NCBI Reference Sequence: NP_776250.2
gi|64762489|ref|NP_776250.2| Znt8 [Homo sapiens]
MEFLERTYLVNDKAAKMYAFTLESVELQQKPVNICDQCPRERPEELESGG
MYHCHSGSKPTEKGANEYAYAKWKLCSASAICFIFMIAEVVGGHIAGSLA
VVTDAAHLLIDLTSFLLSLFSLWLSSKPPSKRLTFGWHRAEILGALLSIL
CIWVVTGVLVYLACERLLYPDYQIQATVMIIVSSCAVAANIVLTVVLHQR
CLGHNHKEVQANASVRAAFVHALGDLFQSISVLISALIIYFKPEYKIADP
ICTFIFSILVLASTITILICDFSILLMEGVPKSLNYSGVKELILAVDGVL
SVHSLHIWSLTMNQVILSAHVATAASRDSQVVRREIAICALSKSFTMHSL
TIQMESPVDQDPDCLFCEDPCD

Region that are aligned after BLAST:

MAP 3865c Length = 296 (residue 2-296)
GAGHNHT---PAETG---DARLIPRMVMAAAILAAFFVVELVTSLLINSI
ALLADAGHMLTDVVAVFMGLAAVTLARRGSSSPARTYGWHRAEVFTAVAN
AGLLIGVSVFILYEAIQRLREAP-AVPGVPMIAVALAGLAANFVVALLLR
SHSSGSLAVKGAYLEVIADTVGSLGVLIAG-VVTVTTRWPYadvvvavlv
alwvlPRAISLARDALRILSESSPTHIDVEELTVQIDCPDDTE-----CS
D
Znt8 Length = 369 (residue 49-366)
GMYHCHSGSKPTEKGANEYAYAKWKLCSASAICFIFMIAEVVGGHIAGSL
AVVTDAAHLLIDLTSFLLSLFSLWLSSKPPSK-RLTFGWHRAEILGALLS
ILCIWVVTGVLVYLACERLLYPDYQIQATVMIIVSSCAVAANIVLTVVLH
QRCLGHNHKEVQANASVRAAFVHALGDLFQSISVLISALIIYFKPEYKIA
DPICTFIFSILVLASTITILKDFSILLMEGVPKSLNYSGVKELILAVDGV
LSVHSLHIWSLTMNQVILSAHVATAASRDSQVVRREIAKALSKSFTMHSL
TIQMESPVDQDPDCLFCED

To further explore the significance of this homology, we focused our analysis on one of the highly conserved regions (41.2% aminoacid identity) corresponding to the MAP3865c₁₂₅₋₁₄₁ and ZnT8₁₇₈₋₁₉₄ sequences. These sequences are located in one of the 6 membrane-spanning domains of the two proteins (FIG. 3B).

Two nonamer peptides covering this region were synthesized: MAP3865c₁₂₅₋₁₃₃ (MIAVALAGL) (SEQ ID NO:1) and MAP3865c₁₃₃₋₁₄₁ (LAANFWAL) (SEQ ID NO:2). Competition assays demonstrated that these epitopes are immunodominant Ab targets within the full-length MAP3865c protein, as sera pre-adsorbed with these peptides, either alone or in combination, were capable of blocking binding to the MAP3865c-MBP fusion protein, to a similar extent to what observed when pre-adsorbing sera with the MAP3865c-MBP protein itself (FIG. 4).

The homologous ZnT8 peptides corresponding to these MAP3865c sequences were further synthesized: ZnT8₁₇₈₋₁₈₆ (MIIVSSCAV) 50 (SEQ ID NO:3) and ZnT8₁₈₆₋₁₉₄ (VAANIVLTV) (SEQ ID NO:4). Serum Ab reactivity against these four MAP3865c and ZnT8 peptides was further tested using the same ELISA assay.

Also in this case, a significant difference in the frequency of Ab+ sera was observed between T1D and healthy subjects (FIG. 5). The homologous MAP3865c₁₂₅₋₁₃₃ and ZnT8₁₇₈₋₁₈₆ peptides (FIG. 5AB) were recognized by 65.4% and 68.0% of T1D patients, but only in 4.2% of healthy controls (AUC 0.85 and 0.86, respectively; p<0.0001 for both). This serum Ab reactivity was also observed for the MAP3865c₁₃₃₋₁₄₁ and ZnT8₁₈₆₋₁₉₄ peptides (FIG. 5C-D), as 51.6% and 55.6% of T1D patients were Ab+, respectively, compared to 4.2% of healthy controls (AUC 0.75 and 0.79; p=0.0003 and p<0.0001, respectively). As observed for the whole MAP3865c protein, this reactivity was specific of T1D patients, as it was not observed among T2D subjects (FIG. 6).

Table 2 shows T1D duration and age at T1D diagnosis in Ab+ and Ab− negative T1D patients. T1D patients whose Ab reactivities are shown in FIGS. 1 and 5 were compared using the Mann-Whitney U test. Mean±SD are shown.

TABLE 2

| | | Age at T1D diagnosis |
|---|---|---|
| | T1D duration (yrs) | (yrs) |
| MAP3865c Ab+ | 10.9 ± 7.7 | 22.9 ± 9.6 |
| MAP3865c Ab− | 17.9 ± 10.0 | 16.3 ± 10.0 |
| p | 0.025 | 0.072 |
| MAP3865c₁₂₅₋₁₃₃ Ab+ | 12.6 ± 8.8 | 19.8 ± 11.0 |
| MAP3865c₁₂₅₋₁₃₃ Ab− | 17.2 ± 10.0 | 17.1 ± 9.8 |
| p | 0.068 | 0.340 |
| ZnT8₁₇₈₋₁₈₆ Ab+ | 12.6 ± 8.8 | 19.8 ± 11.0 |
| ZnT8₁₇₈₋₁₈₆ Ab− | 18.0 ± 10.0 | 16.7 ± 9.8 |
| p | 0.068 | 0.340 |
| MAP3865c₁₃₃₋₁₄₁ Ab+ | 13.2 ± 8.6 | 20.8 ± 10.2 |
| MAP3865c₁₃₃₋₁₄₁ Ab− | 17.5 ± 10.4 | 16.6 ± 10.5 |
| p | 0.170 | 0.229 |
| ZnT8₁₈₆₋₁₉₄ Ab+ | 13.8 ± 12.3 | 20.1 ± 14.3 |
| ZnT8₁₈₆₋₁₉₄ Ab− | 17.5 ± 9.5 | 16.6 ± 9.3 |
| p | 0.296 | 0.347 |

Comparison of Ab+ and Ab− negative T1D patients (Table 2) showed that anti-MAP3865c Ab+patients had significantly shorter disease duration than Ab− negative pairs (10.9±7.7 vs 17.9±10.0; p=0.025). Similar trends were observed when comparing T1D patients harboring or not Abs against MAP3865c₁₂₅₋₁₃₃ (12.6±8.8 vs 17.2±10.0; p=0.068) and its homologous ZnT8₁₇₈₋₁₈₆ (12.6±8.8 vs 18.0±10.0; p=0.068), but not for Abs against MAP3865c₁₃₃₋₁₄₁ (13.2±8.6 vs 17.5±10.4; p=0.170) or its homologous ZnT8₁₈₆₋₁₉₄ (13.8±12.3 vs 17.5±9.5; p=0.296). A trend towards an older age at T1D diagnosis was also observed in patients positive for Abs against MAP3865c (22.9±9.6 vs 16.3±10.0; p=0.072).

Anti-MAP3865c and anti-ZnT8 Abs recognizing homologous sequences are cross-reactive. The similar frequencies of Abs recognizing MAP3865c and ZnT8 homologous regions among T1D patients (65.4-68.0% and 51.6-55.6%, respectively; FIG. 5) suggest that Abs targeting these epitopes could be cross-reactive. Indeed, there was a high degree of correlation between the titers of Abs recognizing MAP3865c and ZnT8 homologous sequences in both T1D patients and healthy controls (FIG. 7A-B; 12=0.74 for MAP3865c₁₂₅₋₁₃₃ vs ZnT8₁₇₈₋₁₈₆ and r₂=0.58 for MAP3865c₁₃₃₋₁₄₁ vs ZnT8₁₈₆₋₁₉₄; p<0.0001). This correlation was maintained when the analysis was restricted to either T1D patients or healthy controls (data not shown). This demonstrates that anti-MAP3865c and anti-ZnT8 Abs recognizing homologous sequences segregate within the same sera. The same was true for Ab reactivities against the two neighboring regions MAP3865c₁₂₅₋₁₃₃ and MAP3865c₁₃₃₋₁₄₁ and for ZnT8₁₇₈₋₁₈₆ and ZnT8₁₈₆₋₁₉₄ (FIG. 7C-D; r₂=0.67 and 0.74, respectively; p<0.0001), suggesting that recognition of these epitopes stems from an immune response against the whole MAP3865c/ZnT8 transmembrane region to which they belong.

To verify whether co-segregation of these reactivities was due to Ab specificities cross-reacting between each other, competition experiments were performed. Anti-MAP3865c₁₂₅₋₁₃₃-positive and negative sera were pre-adsorbed overnight with different peptides, then subjected to ELISA on MAP3865c₁₂₅₋₁₃₃-coated plates (FIG. 8A). While a control peptide did not cause any decrease in signal, both MAP3865c₁₂₅₋₁₃₃ and its homologous ZnT8₁₇₈₋₁₈₆ peptide strongly inhibited the MAP3865c₁₂₅₋₁₃₃ reactivity to a similar extent (57-89%). The same observation was repeated with the MAP3865c₁₃₃₋₁₄₁ reactivity, which was efficiently inhibited (55-66%) upon serum preadsorption with either MAP3865c₁₃₃₋₁₄₁ or its homologous ZnT8₁₈₆₋₁₉₄ (FIG. 8B). Taken together, these results demonstrate that anti-MAP and anti-ZnT8 Abs targeting homologous membrane spanning sequences are cross-reactive.

### EXAMPLE 2

### Comparison Between the Sensitivity of ELISA Test Carried Out by Means the Epitopes of the Present Invention and Known Epitopes in the Diagnosis of T1D Onset

Methods

Subjects

Sardinian T1D patients (n=31; mean age 29.73±8.25 years) diagnosed according to the American Diabetes Association criteria; and Sardinian HCs (n=30; mean age 33.6±7.2) were enrolled in Sassari. Serum samples were processed as previously described.

Peptides

Peptides MAP3865c₁₂₅₋₁₃₃ (MIAVALAGL) (SEQ ID NO:1) and MAP3865c₁₃₃₋₁₄₁ (LAANFVVAL) (SEQ ID NO:2) along with their respective homologous peptides ZnT8₁₇₈₋₁₈₆ (MIIVSSCAV) (SEQ ID NO: 3) and ZnT8₁₈₆₋₁₉₄ (VAANIVLTV) (SEQ ID NO:4); MAP3865c₂₄₆₋₂₅₂ (LSPGKDM) (SEQ ID NO: 9), MAP3865c₂₅₆₋₂₆₂ (HLISTGD) (SEQ ID NO: 10), MAP3865c₂₆₁₋₂₆₇ (GDSARVL) (SEQ ID NO: 11) and MAP3865c₂₈₁₋₂₈₇ (HATVQID) (SEQ ID NO: 12) were synthesized at >85% purity (GL Biochem).

ELISA

ELISA was performed as previously reported. The cutoff for positivity was calculated by ROC analysis, setting specificity at 93.3% (i.e., Ab+ HCs ≦56.7%). The percent fraction of Ab+ sera, Area Under ROC Curve, (AUC) and p values after Fisher exact test are indicated. Results were normalized to a strongly positive control serum included in all experiments, the reactivity of which was set at 10.000 arbitrary units (AU)/ml.

ElisaRSR™ ZnT8 Ab™

ElisaRSR™ ZnT8 Ab™ (RSR Limited Avenue Park Pentwyn, Cardiff, CF23 8HE United Kingdom) kit for the quantitative determination of autoantibodies (aAbs) to the ZnT8 C-terminal region in serum was carried out according to the manufacturer's instructions.

Results

Ab responses against MAP3865c₂₄₆₋₂₅₂, MAP3865c₂₅₆₋₂₆₂, MAP3865c₂₆₁₋₂₆₇ and MAP3865c₂₈₁₋₂₈₇ peptides along with the pairs of homologous peptides ZnT8₁₇₈₋₁₈₆/MAP3865c₁₂₅₋₁₃₃ and ZnT8₁₈₆₋₁₉₄/MAP3865c₁₃₃₋₁₄₁ were analyzed in 31 T1D and 30 HCs. Results are summarized in Table 3.

TABLE 3
Distribution of Antibodies (Abs) against the
peptides MAP3865c₁₂₅₋₁₃₃, Znt8₁₇₈₋₁₈₆, MAP3865C₁₃₃₋₁₄₁,
Znt8₁₈₆₋₁₉₄, MAP3865c₂₄₆₋₂₅₂, MAP3865c₂₅₆₋₂₆₂, MAP3865c₂₆₁₋₂₆₇
and MAP3865c₂₈₁₋₂₈₇ in Sardinian type 1 diabetes
patients (T1D), and healthy controls (HCs).

| | ELISA seroreactivity | | | | |
|---|---|---|---|---|---|
| Peptides | Abs+ (%) | | cut-off | AUC | p-value |
| MAP3865c₁₂₅₋₁₃₃ | | | | | |
| T1D (n = 31) | 22 | (71%) | | 0.89 | P < 0.0001 |
| HCs (n = 30) | 3 | (1%) | 3555.6 | | |
| Znt8₁₇₈₋₁₈₆ | | | | | |
| T1D (n = 31) | 22 | (71%) | | 0.88 | P < 0.0001 |
| HCs (n = 30) | 1 | (3.3%) | 3508 | | |
| MAP3865c₁₃₃₋₁₄₁ | | | | | |
| T1D (n = 31) | 19 | (61.3%) | | 0.89 | 0.0001 |
| HCs (n = 30) | 3 | (10%) | 4298.8 | | |
| Znt8₁₈₆₋₁₉₄ | | | | | |
| T1D (n = 31) | 17 | (54.8%) | | 0.774 | P < 0.0001 |
| HCs (n = 30) | 2 | (6.6%) | 4544.6 | | |
| MAP3865c₂₄₆₋₂₅₂ | | | | | |
| T1D (n = 31) | 20 | (645%) | | 0.947 | P < 0.0001 |
| HCs (n = 30) | 1 | (3.3%) | 5229.8 | | |
| MAP3865c₂₅₆₋₂₆₂ | | | | | |
| T1D (n = 31) | 22 | (71%) | | 0.916 | 0.0003 |
| HCs (n = 30) | 1 | (3.3%) | 4864.7 | | |
| MAP3865c₂₆₁₋₂₆₇ | | | | | |
| T1D (n = 31) | 19 | (61.3%) | | 0.936 | P <0.0001 |
| HCs (n = 30) | 2 | (6.6%) | 5327.5 | | |
| MAP3865c₂₈₁₋₂₈₇ | | | | | |
| T1D (n = 31) | 20 | (64.5%) | | 0.89 | P <0.0001 |
| HCs (n = 30) | 1 | (3.3%) | 4532 | | |

Peptides Belonging to the Fourth Znt8 Transmembrane Domain and MAP3865c Respective Homologues

Ab positivity against MAP3865c₁₂₅₋₁₃₃ was detected in 71% of T1D and only in 1% of HCs (χ Fisher exact test: p<0.0001; AUC=0.89), showing a statistically significant higher frequency in T1D adult (FIG. 9A).

Anti-ZnT8₁₇₈₋₁₈₆ Abs were detected in 71% of T1D and in 1 of HCs (p<0.0001; AUC=0.88 when comparing T1D with HCs (FIG. 9B).

Ab positivity against MAP3865c₁₃₃₋₁₄₁ was detected in 61.3% of T1D and only in 10% of HCs (p=0.0001; AUC=0.89), showing a statistically significant higher frequency in T1D adult (FIG. 9C).

Anti-ZnT8₁₈₆₋₁₉₄Abs were detected in 54.8% of T1D and in 6.6% of HCs (p<0.0001; AUC=0.77 when comparing T1D with HCs) (FIG. 9D).

Peptides belonging to the Znt8 C-terminal Domain (268-369) and MAP3865c respective homologues

MAP3865c₂₄₆₋₂₅₂ Abs reactivity was the same obtained with MAP3865c₂₈₁₋₂₈₇ when comparing T1D with HCs (64.5% and in % 3.3 respectively; P<0.0001; area under ROC curves AUC=0.95 and AUC=0.89) (FIGS. 10A and 10D).

Ab positivity against MAP 3865c₂₅₆₋₂₅₂ was even more remarkable, being detected in 71% of T1D and only 3.3% of HCs (p=0.003; area under ROC curves AUC=0.92) (FIG. 10B).

Ab positivity against MAP 3865c₂₆₁₋₂₆₇ was detected in 61.3% of T1D and only in 6.6% of the HCs (p<0.0001; AUC=0.94), once again the seric Abs reactivity was significantly different between the two groups (FIG. 10C).

Commercially available RSR ZnT8 Ab ELISA kit searches and identifies Abs against residues 275-369 inclusive of the human ZnT8 protein and is also capable of detecting and quantifying, autoantibodies (aAbs) specific to R(Arg) 325 or to W(Trp) 325 variant or to residue Q (Glu) 325 non specific variant (ElisaRSR™ ZnT8 Ab™). MAP 3865c protein sequence was inputted into DNAstar program in order to calculate its antigenic features. Four putatively immunogenic epitopes were identified on the basis of both antigenic index and the probability to be exposed on the surface of the membrane. All of them (Table 4) are homolog to peptides spanning the C-terminal region of human Znt8 and both peptide 52 HLISTGD MAP3865c₂₅₆₋₂₆₂ and peptide 53 GDSARVL MAP3865c₂₆₁₋₂₆₇ fall in the region of homology comprising the polymorphic Znt8 325^{th} residue.

TABLE 4

| | |
|---|---|
| 80412_50 MAP3865c₂₄₆₋₂₅₂ | LSPGKDM |
| 80412_52 MAP3865c₂₅₆₋₂₆₂ | HLISTGD |
| 80412_53 MAP3865c₂₆₁₋₂₆₇ | GDSARVL |
| 80412_57 MAP3865c₂₈₁₋₂₈₇ | HATVQID |

BLAST (MAP3865c from aa 167 to aa 296: SEQ ID NO:15; ZnT8 from aa 228 to aa 366: SEQ ID NO:16)

| | | | |
|---|---|---|---|
| MAP3865c | 167 | SLGVLIAG-VVTVTTRWPYadvvvaylvalwvlPRAISLARDALRILSESSPTHIDVEEL | 225 |
| | | S+ VLI+ ++30 + AD + + ++ VL I++ +D +L E P ++ + | |
| Znt8 | 228 | SISVLISALIIYFKPEYKIADPICTFIFSILVLASTITILKDFSILLMEGVPKSLNYSGV | 287 |
| MAP3865c | 226 | RAALGAVDGVTGVHDLHVWTLSPGKDMCTAHLIS--TGDSARVLRDARAVLSARGLAHA- | 282 |
| | | + + AVDGV VH LH+W+L+ + + +AH+ + + DS V R+ LS H+ | |
| Znt8 | 288 | KELILAVDGVLSVHSLHIWSLTMNQVILSAHVATAASRDSQVVRREIAKALSKSFTMNSL | 347 |

| | | | |
|---|---|---|---|
| MAP3865c | 283 | TVQIDCPDDTE-----CSD | 296 |
| | | T+Q++ P D + C D | |
| Znt8 | 348 | TIQMESPVDQDPDCLFCED | 366 |

Znt8 C terminal Domain (268-369)

D=residue 269 (Znt8)

R=325 (Znt8). Variant R—W or R-Q

52 MAP3865c₂₅₆₋₂₆₂ and 53 MAP3865c₂₆₁₋₂₆₇ for the high antigenic index and for being in correspondence of residue 325

Peptide 50 MAP3865c₂₄₆₋₂₅₂ 57 MAP3865c₂₈₁₋₂₈₇ for the high antigenic index

The specificity of the test was further validated performing the RSR ZnT8 Ab ELISA test in a subset sample of 20 T1D and 19 HC.

After carrying out the RSR ZnT8 Ab ELISA kit (FIG. 11) 6 positives among 20 diabetic patients (30%) were correctly identified.

The identified epitopes were more sensitive in comparison to the ELISA kit (FIGS. 10 and 11) where a range spanning from 17 to 22 diabetic patients (71%) were correctly identified out of 31 patients.

### EXAMPLE 3

### Study on the Recognition of ZnT8 and MAP8635c Homologous Epitopes at T1D Onset in Sardinian Children

Methods

Subjects

Sardinian new-onset T1D children (n=29; mean age 8.6±4 years) diagnosed according to the American Diabetes Association criteria; and Sardinian HCs (n=30; mean age 8±3) were enrolled in Cagliari and Sassari. Serum samples were processed as previously described.

Peptides

Peptides MAP3865c₁₂₅₋₁₃₃ (MIAVALAGL) (SEQ ID NO:1) and MAP3865c₁₃₃₋₁₄₁ (LAANFVVAL) (SEQ ID NO:2) along with their respective homologous peptides ZnT8₁₇₈₋₁₈₆ (MIIVSSCAV) (SEQ ID NO: 3) and ZnT8₁₈₆₋₁₉₄ (VAANIVLTV) (SEQ ID NO:4) were synthesized at >85% purity (GL Biochem).

### ELISA

ELISA was performed as previously reported. The cutoff for positivity was calculated by ROC analysis, setting specificity at 93.3% (i.e., Ab+ HCs 6.7%). The percent fraction of Ab+ sera, Area Under ROC Curve, (AUC) and p values after Fisher exact test are indicated. Results were normalized to a strongly positive control serum included in all experiments, the reactivity of which was set at 10.000 arbitrary units (AU)/ml.

Results

Abs responses against the 8 MAP3865c peptides identified were then analyzed in 29 new-onset T1D children, and 30 age matched HCc using indirect ELISA (FIGS. 12 and 13). Six out of eight peptides were highly recognized showing similar reactivity. Results are summarized in Table 5.

TABLE 5
Distribution of Antibodies (Abs) against the peptides MAP3865c₁₂₅₋₁₃₃,
Znt8₁₇₈₋₁₈₆, MAP3865c₁₃₃₋₁₄₁, Znt8₁₈₆₋₁₉₄, MAP3865c₂₄₆₋₂₅₂,
MAP3865c₂₅₆₋₂₆₂, MAP3865c₂₆₁₋₂₆₇ and MAP3865c₂₈₁₋₂₈₇ in Sardinian type
1 diabetes patients (T1D) children, and age matched healthy controls (HCs).

| | ELISA seroreactivity | | | | |
|---|---|---|---|---|---|
| Peptides | Abs+ (%) | | cut-off | AUC | p-value |
| MAP3865C₁₂₅₋₁₃₃ | | | | | |
| T1D (n = 29) | 6 | (20.7%) | | 0.74 | 0.1455 |
| HCs (n = 30) | 2 | (6.6%) | 7039.5 | | |
| Znt8₁₇₈₋₁₈₅ | | | | | |
| T1D (n = 29) | 6 | (20.7%) | | 0.79 | 0.1455 |
| HCs (n = 30) | 2 | (6.6%) | 6606 | | |
| MAP3865c₁₃₃₋₁₄₁ | | | | | |
| T1D (n = 29) | 12 | (41.4%) | | 0.81 | 0.0021 |
| HCs (n = 30) | 2 | (6.7%) | 7611 | | |
| Znt8₁₈₆₋₁₉₄ | | | | | |
| T1D (n = 29) | 14 | (48.3%) | | 0.82 | 0.0004 |
| HCs (n = 30) | 2 | (6.7%) | 7018.3 | | |
| MAP3865c₂₄₆₋₂₅₂ | | | | | |
| T1D (n = 29) | 8 | (27.6%) | | 0.64 | 0.0122 |
| HCs (n = 30) | 1 | (3.4%) | 6666.2 | | |
| MAP3865c₂₃₆₋₂₆₂ | | | | | |
| T1D (n = 29) | 8 | (27.6%) | | 0.64 | 0.0122 |
| HCs (n = 30) | 1 | (3.4%) | 6926.3 | | |
| MAP3865c₂₆₁₋₂₆₇ | | | | | |
| T1D (n = 29) | 8 | (27.6%) | | 0.69 | 0.0122 |
| HCs (n = 30) | 1 | (3.4%) | 6663 | | |
| MAP3865c₂₈₁₋₂₈₇ | | | | | |
| T1D (n = 29) | 10 | (34.5%) | | 0.7 | 0.0102 |
| HCs (n = 30) | 2 | (7.1%) | 6850.6 | | |

Summing up the data obtained with the 4 peptides spanning MAP3865c₂₄₆₋₂₈₇ region denote a significant humoral response of T1D at disease onset in comparison to healthy children, suggesting that these antibodies could be early biomarkers of T1D.

### BIBLIOGRAPHY

Scotto M, Afonso G, Larger E, Raverdy C, Lemonnier F A, Carel J C, Dubois-Laforgue D, Baz B, Levy D, Gautier J F, Launay O, Bruno G, Boitard C, Sechi L A, Hutton J C, Davidson H W, Mallone R. Zinc transporter (ZnT)8(186-194) is an immunodominant CD8+ T cell epitope in HLA-A2+ type 1 diabetic patients. Diabetologia. 2012 July; 55(7):2026-31. Epub 2012 Apr. 20.
Masala S, Paccagnini D, Cossu D, Brezar V, Pacifico A, Ahmed N, Mallone R, Sechi L A. Antibodies recognizing Mycobacterium avium paratuberculosis epitopes cross-react with the beta-cell antigen ZnT8 in Sardinian type 1 diabetic patients. PLoS One. 2011; 6(10):e26931. Epub 2011 Oct. 27.
Yu L, Boulware D C, Beam C A, Hutton J C, Wenzlau J M, Greenbaum C J, Bingley P J, Krischer J P, Sosenko J M, Skyler J S, Eisenbarth G S, Mahon J L; Type 1 Diabetes TrialNet Study Group.Zinc transporter-8 autoantibodies improve prediction of type 1 diabetes in relatives positive for the standard biochemical autoantibodies. Diabetes Care. 2012 June; 35(6):1213-8. Epub 2012 Mar. 23.
Wenzlau J M, Juhl K, Yu L, Moua O, Sarkar S A, Gottlieb P, Rewers M, Eisenbarth G S, Jensen J, Davidson H W, Hutton J C. The cation efflux transporter ZnT8 (Slc30A8) is a major autoantigen in human type 1 diabetes. Proc Natl Acad Sci USA. 2007 Oct. 23; 104(43):17040-5. Epub 2007 Oct. 17.
Achenbach P, Lampasona V, Landherr U, Koczwara K, Krause S, Grallert H, Winkler C, Pflger M, Illig T, Bonifacio E, Ziegler A G. Autoantibodies to zinc transporter 8 and SLC30A8 genotype stratify type 1 diabetes risk. Diabetologia. 2009 September; 52(9):1881-8. Epub 2009 Jul. 10.
Wenzlau J M, Walter M, Gardner T J, Frisch L M, Yu L, Eisenbarth G S, Ziegler A G, Davidson H W, Hutton J C. Kinetics of the post-onset decline in zinc transporter 8 autoantibodies in type 1 diabetic human subjects. J Clin Endocrinol Metab. 2010 October; 95(10):4712-9. Epub 2010 Jul. 7.

1. A method for diagnosing an individual who is susceptible to or who is developing type I diabetes, the method comprising
contacting a sample from the individual with at least one isolated peptide belonging to MAP3865c or ZnT8, or at least one isolated antibody specific for said at least one peptide, in an in vitro test
wherein said at least one peptide is chosen from the group consisting of MAP3865c125-133 having sequence MIAVALAGL (SEQ ID NO:1), MAP3865c133-141 having sequence LAANFVVAL (SEQ ID NO:2), MAP3865c121-127 having sequence PGVPMIA (SEQ ID NO:13), MAP3865c131-137 having sequence AGLAANF (SEQ ID NO:14), MAP3865c₂₄₆₋₂₅₂ having sequence LSPGKDM (SEQ ID NO:9), MAP3865c₂₅₆₋₂₆₂ having sequence HLISTGD (SEQ ID NO:10), MAP3865c₂₆₁₋₂₆₇ having sequence GDSARVL (SEQ ID NO:11), MAP3865c₂₈₁₋₂₈₇ having sequence HATVQID (SEQ ID NO:12), ZnT8178-186 having sequence MIIVSSCAV (SEQ ID NO:3), ZnT8186-194 having sequence VAANIVLTV (SEQ ID NO:4),
wherein the at least one isolated peptide belonging to MPA 3865c or the at least one isolated antibody specific for said at least one peptide are detected as biomarker for diagnosing the individual who is susceptible to or who is developing type I diabetes.

2. (canceled)

3. (canceled)

4. (canceled)

5. (canceled)

6. The method according to claim 1, wherein the peptides are at least the following four peptides: MAP3865c125-133 having sequence MIAVALAGL (SEQ ID NO:1), MAP3865c133-141 having sequence LAANFVVAL (SEQ ID NO:2), and their respective homologous peptides ZnT8178-186 having sequence MIIVSSCAV (SEQ ID NO:3), ZnT8186-194 having sequence VAANIVLTV (SEQ ID NO:4).

7. The method according to claim 1, wherein the peptides are at least the following three peptides MAP3865c125-133 having sequence MIAVALAGL (SEQ ID NO:1), MAP3865c₂₈₁₋₂₈₇ having sequence HATVQID (SEQ ID NO:12), ZnT8178-186 having sequence MIIVSSCAV (SEQ ID NO:3).

8. The method Use according claim 1, wherein the peptides are all the following ten peptides: MAP3865c125-133 having sequence MIAVALAGL (SEQ ID NO:1), MAP3865c133-141 having sequence LAANFVVAL (SEQ ID NO:2), ZnT8178-186 having sequence MIIVSSCAV (SEQ ID NO:3), ZnT8186-194 having sequence VAANIVLTV (SEQ ID NO:4), MAP3865c121-127 having sequence PGVPMIA (SEQ ID NO:13), MAP3865c131-137 having sequence AGLAANF (SEQ ID NO:14), MAP3865c₂₄₆₋₂₅₂ having sequence LSPGKDM (SEQ ID NO:9), MAP3865c₂₅₆₋₂₆₂ having sequence HLISTGD (SEQ ID NO:10), MAP3865c₂₆₁₋₂₆₇ having sequence GDSARVL (SEQ ID NO:11), MAP3865c₂₈₁₋₂₈₇ having sequence HATVQID (SEQ ID NO:12).

9. A method for in vitro diagnosing a subject who is susceptible to or who is developing type I diabetes, said method comprising:
a) detecting and quantifying, in a blood sample of said subject, antibodies specific for at least one peptide belonging to MAP3865c or ZnT8, to obtain detected and quantified values, wherein said at least one peptide is chosen from the group consisting of MAP3865c125-133 having sequence MIAVALAGL (SEQ ID NO:1), MAP3865c133-141 having sequence LAANFVVAL (SEQ ID NO:2), MAP3865c121-127 having sequence PGVPMIA (SEQ ID NO:13), MAP3865c131-137 having sequence AGLAANF (SEQ ID NO:14), MAP3865c₂₄₆₋₂₅₂ having sequence LSPGKDM (SEQ ID NO:9), MAP3865c₂₅₆₋₂₆₂ having sequence HLISTGD (SEQ ID NO:10), MAP3865c₂₆₁₋₂₆₇ having sequence GDSARVL SEQ ID NO:11 MAP3865c₂₈₁₋₂₈₇ having sequence HATVQID (SEQ ID NO:12), ZnT8178-186 having sequence MIIVSSCAV (SEQ ID NO:3), ZnT8186-194 having sequence VAANIVLTV (SEQ ID NO:4); and
b) comparing the detected and quantified values of step a) with those of an healthy control.

10. (canceled)

11. (canceled)

12. The method according to claim 9, wherein the peptides are at least the following four peptides: MAP3865c125-133 having sequence MIAVALAGL (SEQ ID NO:1), MAP3865c133-141 having sequence LAANFVVAL (SEQ ID NO:2), and their respective homologous peptides ZnT8178-186 having sequence MIIVSSCAV (SEQ ID NO:3), ZnT8186-194 having sequence VAANIVLTV (SEQ ID NO:4).

13. The method according to claim 9, wherein the peptides are at least the following three peptides: MAP3865c125-133 having sequence MIAVALAGL (SEQ ID NO:1), MAP3865c₂₈₁₋₂₈₇ having sequence HATVQID (SEQ ID NO:12), ZnT8178-186 having sequence MIIVSSCAV (SEQ ID NO:3).

14. The method according to claim 9, wherein the peptides are all the following ten peptides: MAP3865c125-133 having sequence MIAVALAGL (SEQ ID NO:1), MAP3865c133-141 having sequence LAANFVVAL (SEQ ID NO:2), ZnT8178-186 having sequence MIIVSSCAV (SEQ ID NO:3), ZnT8186-194 having sequence VAANIVLTV (SEQ ID NO:4), MAP3865c121-127 having sequence PGVPMIA (SEQ ID NO:13), MAP3865c131-137 having sequence AGLAANF (SEQ ID NO:14), MAP3865c₂₄₆₋₂₅₂ having sequence LSPGKDM (SEQ ID NO:9), MAP3865c₂₅₆₋₂₆₂ having sequence HLISTGD (SEQ ID NO:10), MAP3865c₂₆₁₋₂₆₇ having sequence GDSARVL (SEQ ID NO:11), MAP3865c₂₈₁₋₂₈₇ having sequence HATVQID (SEQ ID NO:12).

15. The method according to claim 9, wherein said method is carried out by ELISA.

16. A method for in vitro diagnosing a subject who is susceptible to or who is developing type I diabetes,
said method comprising incubating a blood sample comprising lymphocytes from said subject in the presence of
at least one peptide belonging to MAP3865c, said at least one MAP3865c peptide having an homology of at least 50% in comparison to a corresponding peptide belonging to human ZnT8 sequence after optimal alignment;
and/or in the presence of at least one peptide belonging to ZnT8 sequence from aminoacid 174 to aminoacid 194, said ZnT8 peptide having an homology of at least 80% in comparison to a corresponding peptide belonging to MAP3865c from aminoacid 121 to aminoacid 141,
the incubating performed for a time and under conditions sufficient to stimulate the lymphocytes to produce an effector molecule, wherein the presence or level of the effector molecule is indicative of the lymphocytes derived from a subject susceptible to or who is developing type I diabetes
wherein said at least one peptide belonging to MAP3865c is chosen from the group consisting of MAP3865c125-133 having sequence MIAVALAGL (SEQ ID NO:1), MAP3865c133-141 having sequence LAANFVVAL (SEQ ID NO:2), MAP3865c121-127 having sequence PGVPMIA (SEQ ID NO:13), MAP3865c131-137 having sequence AGLAANF (SEQ ID NO:14), MAP3865c₂₄₆₋₂₅₂ having sequence LSPGKDM (SEQ ID NO:9), MAP3865c₂₅₆₋₂₆₂ having sequence HLISTGD SEQ ID NO:10 MAP3865c₂₆₁₋₂₆₇ having sequence GDSARVL (SEQ ID NO:11), MAP3865c₂₈₁₋₂₈₇ having sequence HATVQID (SEQ ID NO:12), ZnT8178-186 having sequence MIIVSSCAV (SEQ ID NO:3), ZnT8186-194 having sequence VAANIVLTV (SEQ ID NO:4).

17. (canceled)

18. (canceled)

19. The method according to claim 16, wherein the peptides are at least the following four peptides: MAP3865c125-133 having sequence MIAVALAGL (SEQ ID NO:1), MAP3865c133-141 having sequence LAANFVVAL (SEQ ID NO:2) and their respective homologous peptides ZnT8178-186 having sequence MIIVSSCAV (SEQ ID NO:3), ZnT8186-194 having sequence VAANIVLTV (SEQ ID NO:4).

20. The method according to claim 16, wherein the peptides are at least the following three peptides: MAP3865c125-133 having sequence MIAVALAGL (SEQ ID NO:1), MAP3865c₂₈₁₋₂₈₇ having sequence HATVQID (SEQ ID NO:12), ZnT8178-186 having sequence MIIVSSCAV (SEQ ID NO:3).

21. The method according to claim 16, wherein the peptides are all the following ten peptides: MAP3865c125-133 having sequence MIAVALAGL (SEQ ID NO:1), MAP3865c133-141 having sequence LAANFVVAL (SEQ ID NO:2), ZnT8178-186 having sequence MIIVSSCAV (SEQ ID NO:3), ZnT8186-194 having sequence VAANIVLTV (SEQ ID NO:4), MAP3865c121-127 having sequence PGVPMIA (SEQ ID NO:13), MAP3865c131-137 having sequence AGLAANF (SEQ ID NO:14), MAP3865c₂₄₆₋₂₅₂ having sequence LSPGKDM (SEQ ID NO:9), MAP3865c₂₅₆₋₂₆₂ having sequence HLISTGD (SEQ ID NO:10), MAP3865c₂₆₁₋₂₆₇ having sequence GDSARVL (SEQ ID NO:11), MAP3865c₂₈₁₋₂₈₇ having sequence HATVQID (SEQ ID NO:12).

22. The method according to claim 16, wherein the effector molecule is selected from interferon-γ, a cytokine, an interleukin and/or TNF-α.

23. An isolated peptide belonging to MAP3865c, said MAP3865c peptide having an homology of at least 50% in comparison to a corresponding peptide belonging to human ZnT8 sequence after optimal alignment; or belonging to ZnT8 sequence from aminoacid 174 to aminoacid 194, said ZnT8 peptide having an homology of at least 80% in comparison to a corresponding peptide belonging to MAP3865c from aminoacid 121 to aminoacid 141,
wherein said isolated peptide belongs to MAP3865c from aminoacid 121 to aminoacid 141,

24. (canceled)

25. The isolated peptide according to claim 23, wherein said peptide is chosen from the group consisting of MAP3865c125-133 having sequence MIAVALAGL (SEQ ID NO:1), MAP3865c133-141 having sequence LAANFVVAL (SEQ ID NO:2), MAP3865c121-127 having sequence PGVPMIA (SEQ ID NO:13), MAP3865c131-137 having sequence AGLAANF (SEQ ID NO:14), MAP3865c₂₄₆₋₂₅₂ having sequence LSPGKDM (SEQ ID NO:9), MAP3865c₂₅₆₋₂₆₂ having sequence HLISTGD (SEQ ID NO:10), MAP3865c₂₆₁₋₂₆₇ having sequence GDSARVL (SEQ ID NO:11), MAP3865c₂₈₁₋₂₈₇ having sequence HATVQID (SEQ ID NO:12), ZnT8178-186 having sequence MIIVSSCAV (SEQ ID NO:3), ZnT8186-194 having sequence VAANIVLTV (SEQ ID NO:4).

26. An isolated nucleic acid molecule encoding for the peptide as defined in claim 23.

27. A vector comprising the nucleic acid molecule according to claim 26.

28. An isolated cell comprising the vector according to claim 27.

29. A kit comprising a container, said container comprising at least one peptide according to claim 23 or at least one nucleic acid molecule encoding for the peptide as defined according to claim 23.

30. The kit according to claim 28, wherein the peptides are at least the following four peptides: MAP3865c125-133 having sequence MIAVALAGL (SEQ ID NO:1), MAP3865c133-141 having sequence LAANFVVAL (SEQ ID NO:2), and their respective homologous peptides ZnT8178-186 having sequence MIIVSSCAV (SEQ ID NO:3), ZnT8186-194 having sequence VAANIVLTV (SEQ ID NO:4).

31. The kit according to claim 29, wherein the peptides are at least the following three peptides: MAP3865c125-133 having sequence MIAVALAGL (SEQ ID NO:1), MAP3865c281-287 having sequence HATVQID (SEQ ID NO:12), ZnT8178-186 having sequence MIIVSSCAV (SEQ ID NO:3).

32. The kit according to claim 29, wherein the peptides are all the following ten peptides: MAP3865c125-133 having sequence MIAVALAGL (SEQ ID NO:1), MAP3865c133-141 having sequence LAANFVVAL (SEQ ID NO:2), ZnT8178-186 having sequence MIIVSSCAV (SEQ ID NO:3), ZnT8186-194 having sequence VAANIVLTV (SEQ ID NO:4), MAP3865c121-127 having sequence PGVPMIA (SEQ ID NO:13), MAP3865c131-137 having sequence AGLAANF (SEQ ID NO:14), MAP3865c246-252 having sequence LSPGKDM (SEQ ID NO:9), MAP3865c₂₅₆₋₂₆₂ having sequence HLISTGD (SEQ ID NO:10), MAP3865c₂₆₁₋₂₆₇ having sequence GDSARVL (SEQ ID NO:11), MAP3865c₂₈₁₋₂₈₇ having sequence HATVQID (SEQ ID NO:12).

33. An isolated antibody specific for the peptide as defined in claim 23.

34. A vaccine for treatment or prophylaxis of type I diabetes, said vaccine comprising at least one isolated peptide as defined in claim 23.

35. The vaccine according to claim 34, wherein the peptides are at least the following four peptides: MAP3865c125-133 having sequence MIAVALAGL (SEQ ID NO:1), MAP3865c133-141 having sequence LAANFVVAL (SEQ ID NO:2), and their respective homologous peptides ZnT8178-186 having sequence MIIVSSCAV (SEQ ID NO:3), ZnT8186-194 having sequence VAANIVLTV (SEQ ID NO:4).

36. The vaccine according to claim 34, wherein the peptides are at least the following three peptides: MAP3865c125-133 having sequence MIAVALAGL (SEQ ID NO:1), MAP3865c₂₈₁₋₂₈₇ having sequence HATVQID (SEQ ID NO:12), ZnT8178-186 having sequence MIIVSSCAV (SEQ ID NO:3).

37. The vaccine according to claim 34, wherein the peptides are all the following ten peptides: MAP3865c125-133 having sequence MIAVALAGL (SEQ ID NO:1), MAP3865c133-141 having sequence LAANFVVAL (SEQ ID NO:2), ZnT8178-186 having sequence MIIVSSCAV (SEQ ID NO:3), ZnT8186-194 having sequence VAANIVLTV (SEQ ID NO:4), MAP3865c121-127 having sequence PGVPMIA (SEQ ID NO:13), MAP3865c131-137 having sequence AGLAANF (SEQ ID NO:14), MAP3865c₂₄₆₋₂₅₂ having sequence LSPGKDM (SEQ ID NO:9), MAP3865c₂₅₆₋₂₆₂ having sequence HLISTGD (SEQ ID NO:10), MAP3865c₂₆₁₋₂₆₇ having sequence GDSARVL (SEQ ID NO:11), MAP3865c₂₈₁₋₂₈₇ having sequence HATVQID (SEQ ID NO:12).

38. A method for prevention and treatment of type I diabetes, in an individual, the method comprising
administering to the individual an effective amount of one or more anti Mycobacterium avium paratuberculosis drugs.

39. The method according to claim 38, wherein said drugs are chosen from the group consisting of clarithromycin, rifabutin, clofazimine.
